(19) United States
(12) Patent Application Publication
(10) Pub. No.: US 2020/0321089 A1
(43) Pub. Date: Oct. 8, 2020

(54) BIOLOGICAL INFORMATION PROCESSING DEVICE, PROCESSING METHOD, AND NON-TRANSITORY RECORDING MEDIUM FOR RECORDING PROCESSING PROGRAM

(71) Applicant: OMRON HEALTHCARE Co., Ltd., Kyoto (JP)

(72) Inventors: Hiroshi NAKAJIMA, Kyoto-shi (JP); Hirotaka WADA, Kyoto-shi (JP); Hironori SATO, Kyoto (JP); Taiji YOSHIKAWA, Kyoto-shi (JP); Tamio UEDA, Kyoto-shi (JP)

(21) Appl. No.: 16/906,368

(22) Filed: Jun. 19, 2020

Related U.S. Application Data
(63) Continuation of application No. PCT/JP2018/046246, filed on Dec. 17, 2018.

(30) Foreign Application Priority Data
Dec. 27, 2017 (JP) 2017-252564

(51) Int. Cl. G16H 10/60; A61B 5/00; A61B 5/022; A61B 5/11

(52) U.S. Cl. CPC G16H 10/60; A61B 5/742; A61B 5/022; A61B 2562/029; A61B 5/681; A61B 2562/0219; A61B 2562/0271; A61B 5/1118

(57) ABSTRACT

Provided is a technology that enables biological information relating to a subject to be processed appropriately and independently of on the subject. A biological information processing device according to the present embodiment includes a storage device, an estimation unit, an acquisition unit, and a correction unit. The storage unit stores biological information relating to a specified subject for a unit period including a plurality of times. The estimation unit estimates variable characteristics of the biological information based on the stored biological information relating to the specified subject. The acquisition unit acquires the biological information relating to the specified subject together with information indicating a measurement time of the biological information. The correction unit corrects the acquired biological information relating to the specified subject based on the information indicating the measurement time, which is acquired together with the biological information, and the estimated variable characteristics.

### CROSS-REFERENCE TO RELATED APPLICATIONS

This is a continuation of International Application 2017-252564, with an international filing date of Dec. 27, 2017, and International Application PCT/JP2018/046246, with an international filing date of Dec. 17, 2018 filed by applicant, the disclosure of which is hereby incorporated by reference in its entirety.

### TECHNICAL FIELD

The present invention relates to a biological information processing device, a processing method, and a non-transitory recording medium for recording a processing program.

### BACKGROUND ART

A blood pressure value is one example of biological information. The blood pressure value is one of the important indexes for determining a health state of a subject. Thus, the blood pressure value is reflected in various types of information that are derived from the health state of the user.

JP 2001-249996 A proposed a home doctor center system in which physical condition data including a beat rate and a blood pressure value that are measured from a member is transmitted to a home doctor center and a physical condition of the member is primarily diagnosed based on the transmitted physical condition data. According to the home doctor center system, a health state can be diagnosed based on the physical condition data such as a blood pressure value of the member, and the result can be utilized for various types of accessorial services.

### SUMMARY OF INVENTION

However, a measurement value of biological information such as a blood pressure value is likely to vary due to external causes such as environment, and hence caution is required when biological information such as a measured blood pressure value is utilized for accessorial services. For example, it is known that the blood pressure value may vary depending on a measurement time and that variable characteristics of a blood pressure value depending on the measurement time differ among individuals. Thus, even when measurement is performed for a plurality of individuals having different health states at the same measurement time, blood pressure values at the same degree may be obtained. Therefore, it can be predicted that, when a measured blood pressure value is utilized as it is for services without considering differences of variable characteristics among subjects, some subjects may not be able to receive desired services depending on a measurement time.

An aspect of the present invention has been made in view of the above-mentioned circumstances, and has an object to provide a technology that enables biological information relating to a subject to be processed appropriately and independently of the subject.

The present invention adopts the following configurations in order to achieve the above-mentioned object.

Specifically, a biological information processing device according to an aspect of the present invention includes a storage device configured to store biological information relating to a specified subject during a unit period including a plurality of times, an estimation unit configured to estimate variable characteristics of the biological information based on the biological information relating to the specified subject that is stored, an acquisition unit configured to acquire the biological information relating to the specified subject together with information indicating a measurement time of the biological information, and a correction unit configured to correct the biological information relating to the specified subject that is acquired, based on the information indicating the measurement time that is acquired together with the biological information and the variable characteristics that are estimated.

With this configuration, the estimation information is estimated based on the plurality pieces of biological information measured at the different times. Thus, based on the discrete biological information, the variable characteristics of the biological information continuous over the entire period can be grasped. Note that the estimation information is estimated from the biological information relating to the same subject. Thus, even when the variable characteristics are specific to the subject, which cannot be deemed as variable characteristics of other subjects, the variation amount of the biological information can be estimated as appropriate. Therefore, a technology that enables the biological information relating to the subject to be processed appropriately and independently of the subject can be provided.

The biological information processing device according to the aspect may further include a generation unit configured to generate analysis information relating to the biological information relating to the specified subject, based on the biological information that is corrected by the correction unit and the variable characteristics that are estimated. With this configuration, the information that enables the relationship between the corrected biological information and the variable characteristics to be grasped can be generated. Thus, at the time of evaluating the biological information, the more useful information can be provided.

In the biological information processing device according to the aspect, the estimation unit may be configured to update the variable characteristics that are estimated, based on the biological information relating to the specified subject that is acquired. With this configuration, at the time of estimating the variable characteristics, the latest biological information can be taken in. With this, even when the variable characteristics of the biological information changes as the health state of the subjects slightly changes, the change can be followed, and thus the biological information can be corrected appropriately.

In the biological information processing device according to the aspect, the estimation unit may be configured to estimate the variable characteristics of the biological information relating to the specified subject, further based on statistic information relating to variable characteristics of biological information relating to at least one unspecified subject during a unit time including a plurality of times. With this configuration, the biological information from the specified subject can be supplemented with the statistic variable characteristics. With this, when the variable characteristics cannot be estimated accurately only based on the biological information from the specified subject, interpolation can be performed through use of a rough variable characteristic trend of the general variable characteristics. Therefore, the biological information relating to the subject can be corrected without being deviated largely from the variable characteristics.

In the biological information processing device according to the aspect, the estimation unit may be configured to estimate a plurality of variable characteristics corresponding to different periods in the biological information, based on the biological information relating to the specified subject that is stored. With this configuration, when the plurality of variable characteristics that vary cyclically during different periods overlap in the biological information, each of the plurality of variable characteristics can be extracted. With this, each of the plurality of cyclically variable components included in the biological information can be eliminated. Therefore, the biological information relating to the subject can be processed more appropriately.

In the biological information processing device according to the aspect, the unit period may be set to any one of a year, a week, and a day. With this configuration, various cyclic variations in the biological information can be taken into consideration. With this, the biological information relating to the subject can be processed more appropriately.

The biological information processing device according to the aspect may further include an evaluation unit configured to generate evaluation information corresponding to a health state of the specified subject, based on the biological information that is corrected by the correction unit. With this configuration, the evaluation unit can evaluate the health state of the specified subject based on the biological information from which an impact of variation due to the different measurement times is eliminated.

In the biological information processing device according to the aspect, the evaluation information may include at least one of information for determining acceptance of insurance purchase and information for calculating an insurance premium or reward points. With this configuration, based on the biological information, the evaluation result of the biological information relating to the subject can be reflected in acceptance of insurance purchase, an insurance premium, or reward points. With this, the subject can purchase appropriate insurance, pay an appropriate insurance premium, or receive an appropriate coupon as a result of fair evaluation without being affected by a measurement time.

According to the present invention, the technology that enables the biological information relating to the subject to be processed appropriately and independently of the subject can be provided.

### BRIEF DESCRIPTION OF DRAWINGS

FIG. 1 is a block diagram illustrating a functional configuration of a biological information processing device according to an application example.

FIG. 2 is a schematic diagram illustrating a configuration of a blood pressure processing system including a blood pressure processing device according to the present embodiment.

FIG. 3 is a block diagram illustrating a hardware configuration of a blood pressure measurement device according to the present embodiment.

FIG. 4 is a block diagram illustrating a hardware configuration of a portable terminal according to the present embodiment.

FIG. 5 is a block diagram illustrating a hardware configuration of a blood pressure processing device according to the present embodiment.

FIG. 6 is a diagram illustrating variable characteristic estimation information according to the present embodiment.

FIG. 7 is a block diagram illustrating a hardware configuration of a blood pressure evaluation device according to the present embodiment.

FIG. 8 is a block diagram illustrating a functional configuration of the blood pressure processing device according to the present embodiment.

FIG. 9 is a diagram illustrating a relationship between blood pressure change information and variable characteristic estimation information according to the present embodiment.

FIG. 10 is a block diagram illustrating a functional configuration of the blood pressure evaluation device according to the present embodiment.

FIG. 11 is a flowchart illustrating a procedure of a blood pressure analysis process in the blood pressure processing device according to the present embodiment.

FIG. 12 is a flowchart illustrating a procedure of a blood pressure evaluation process in the blood pressure evaluation device according to the present embodiment.

FIG. 13 is a diagram illustrating effects according to the present embodiment.

FIG. 14 is a diagram illustrating the variable characteristic estimation information according to the present embodiment in a first modified example.

FIG. 15 is a diagram illustrating the variable characteristic estimation information according to the present embodiment in the first modified example.

FIG. 16 is a block diagram illustrating a functional configuration of the blood pressure processing device according to the present embodiment in a third modified example.

FIG. 17 is a diagram illustrating evaluation reference information according to the present embodiment in a fourth modified example.

### DESCRIPTION OF EMBODIMENTS

Now, with reference to the drawings, embodiments are described. Note that, in the following description, constituent elements having the same function and configuration are denoted with a shared reference symbol. Further, when a plurality of constituent elements having a shared reference symbol are distinguished from one another, distinction is made by adding additional symbols following the shared reference symbol. Note that, when there is no particular need in distinguishing a plurality of constituent components, the plurality of constituent components are denoted only with a shared reference symbol without an additional symbol.

### 1. Application Example

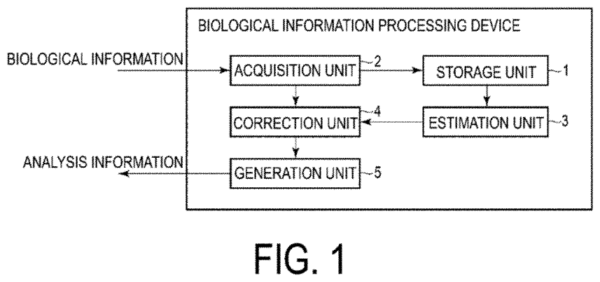

First, with reference to FIG. 1, an example of a biological information processing device to which the present invention is applied is described.

As illustrated in FIG. 1, the biological information processing device includes a storage unit 1, an acquisition unit 2, an estimation unit 3, a correction unit 4, and a generation unit 5.

The storage unit 1 stores biological information relating to a specified subject for a unit period including a plurality of times.

The acquisition unit 2 acquires the biological information relating to the specified subject being a measurement target together with information indicating a measurement time within the unit period of the biological information. For example, the acquisition unit 2 acquires, from a sensor, the biological information such as a blood pressure value of the subject together with the information indicating the measurement date/time.

The estimation unit 3 estimates variable characteristics of the biological information based on the stored biological information relating to the specified subject. The variable characteristics are information indicating an extent at which the biological information varies at the times within the unit period. In this case, a larger number of pieces of biological information stored in the storage unit 1 can improve accuracy of estimation of the variable characteristics.

The correction unit 4 corrects the acquired biological information based on the information indicating the measurement time of the biological information, which is acquired at the same timing, and the variable characteristics estimated by the estimation unit 3. For example, the correction unit 4 extracts, from the estimated variable characteristics, a variation amount at a time corresponding to the measurement date/time of the biological information acquired by the acquisition unit 2, and corrects the acquired biological information in accordance with the extracted variation amount.

The generation unit 5 generates analysis information relating to the biological information relating to the specified subject based on the biological information corrected by the correction unit 4 and the variable characteristics estimated by the estimation unit 3. The analysis information includes information, which is obtained by combining the corrected biological information with the estimated variable characteristics, in addition to the corrected biological information. For example, the analysis information includes information indicating an extent to which the biological information relating to the subject may vary about the corrected biological information.

With the configuration described above, even when the biological information relating to the subject includes a variable component depending on the measurement time, an impact given by the variable component can be suppressed. Specifically, the biological information relating to the subject can be processed appropriately and independently of the measurement time. Further, the biological information can be evaluated while suppressing an impact given by the variable component. In addition, even when the variable characteristics of the blood pressure value of the subject exhibit behavior different from generally-observed variable characteristics of a blood pressure value, the blood pressure value of the subject can be evaluated appropriately.

### 2. Examples

Examples of the biological information processing device according to the application example described above are described below. In the following, a blood pressure processing system including a blood pressure processing device is described as an example of the biological information processing device.

### 2.1 Overall Configuration Example

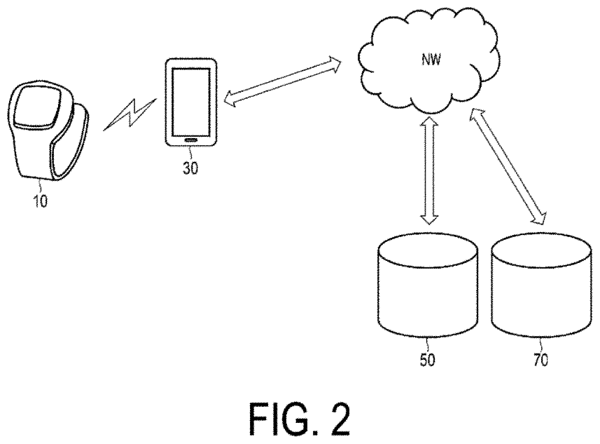

FIG. 2 schematically illustrates an example of an application scene of the blood pressure processing system according to the present embodiment. The blood pressure processing system according to the present embodiment is a system that provides an evaluation result in accordance with a health state of a subject by processing a blood pressure value measured from the subject into a format that is independent of a measurement time. The health state of the subject is associated with a health risk that the subject faces. The risk in the present embodiment indicates an onset risk for cerebrovascular and cardiovascular diseases.

As illustrated in FIG. 2, the blood pressure processing system includes a blood pressure measurement device 10, a portable terminal 30, a blood pressure processing device 50, and a blood pressure evaluation device 70. The blood pressure measurement device 10 and the blood pressure processing device 50 are communicably connected to each other via the portable terminal 30. Further, the portable terminal 30 is connected to the blood pressure processing device 50 and the blood pressure evaluation device 70 via a network NW.

For example, the blood pressure measurement device 10 is a wearable device to be worn at a freely-selected measurement part (for example, a wrist), which is given by a manager of the blood pressure evaluation device 70 and is carried by the subject. The blood pressure measurement device 10 measures a blood pressure value of the subject at the measurement part, and generates blood pressure information in which the blood pressure value is associated with measurement date/time. The measurement date/time is information for specifying a time within a cyclic unit period, and is an example of “time” in the present invention. In the following description, the “unit period” is also simply referred to as a “period”. The cyclic period includes a day, a week, and a year, for example. A time within the cyclic period includes hours in a day, days in a week, and months in a year, for example. The blood pressure measurement device 10 transmits the blood pressure information to the portable terminal 30.

For example, the portable terminal 30 is a terminal that can be carried by the subject. When receiving the blood pressure information from the blood pressure measurement device 10, the portable terminal 30 transmits the blood pressure information to the blood pressure processing device 50. Further, when receiving evaluation information indicating an evaluation result of a blood pressure value from the blood pressure processing device 50, the portable terminal 30 notifies the subject of the evaluation result.

For example, the blood pressure processing device 50 is a server device including a functional configuration for evaluating a blood pressure value of the subject. The blood pressure processing device 50 associates a plurality pieces of blood pressure information from the same subject with each other, and stores the information as blood pressure change information. The blood pressure change information is an example of “biological information relating to the specific subject during the unit period including a plurality of times” in the present invention. The blood pressure processing device 50 estimates variable characteristic estimation information, which indicates cyclic variation of the blood pressure value of the subject, based on the blood pressure change information. The variable characteristic estimation information is an example of “estimated variable characteristics” in the present invention. The blood pressure processing device 50 further acquires the blood pressure information relating to the subject. The blood pressure processing device 50 considers a cyclic variation amount of the further acquired blood pressure information at the measurement date/time based on the variable characteristic estimation information, and thus corrects the blood pressure value so as to eliminate an impact of the cyclic variation. The blood pressure processing device 50 generates analysis information relating to the blood pressure value of the subject based on the corrected blood pressure value, and transmits the information to the blood pressure evaluation device 70. For example, the analysis information includes information on a variation range of the blood pressure value of the subject, and a maximum value, a minimum value, and a most frequent value of the variation, in addition to the corrected blood pressure value.

For example, the blood pressure evaluation device 70 is a server device of an insurance company or the like, which includes a functional configuration for evaluating the blood pressure value of the subject. The blood pressure evaluation device 70 generates evaluation information based on the analysis information, and transmits the information to the portable terminal 30. The evaluation information includes a freely-selected mode that is derived from a health state of the subject. For example, the evaluation information includes information for determining whether the subject is allowed to purchase desired insurance and basic information for calculating an insurance premium of health insurance that the subject purchases or reward points such as a coupon that is given in accordance with a health state of the subject.

With this, the blood pressure processing device 50 can generate the analysis information in accordance with the health state of the subject without being impacted by the cyclic variation amount of the blood pressure value. Further, the blood pressure evaluation device 70 can notify the subject of the evaluation information based on the analysis information.

As described above, in the present embodiment, the blood pressure processing device 50 estimates the variable characteristics of the blood pressure value of the subject as the variable characteristic estimation information based on change of the blood pressure value of the same subject during the period. The variable characteristic estimation information is used dedicatedly for correcting the blood pressure value of the subject. Thus, the blood pressure processing device 50 can process the blood pressure value of the subject appropriately even when the variable characteristics of the blood pressure value of the subject exhibit behavior different from generally-observed variable characteristics of a blood pressure value. For example, when blood pressure values at the same degree are measured from two subjects at the same time, the blood pressure processing device 50 may be capable of generating analysis information that can evaluate that the two subjects have different risks.

### 2.2 Hardware Configuration Examples

An example of a hardware configuration of each of the devices of the blood pressure processing system according to the present embodiment is described.

### 2.2.1 Hardware Configuration of Blood Pressure Measurement Device

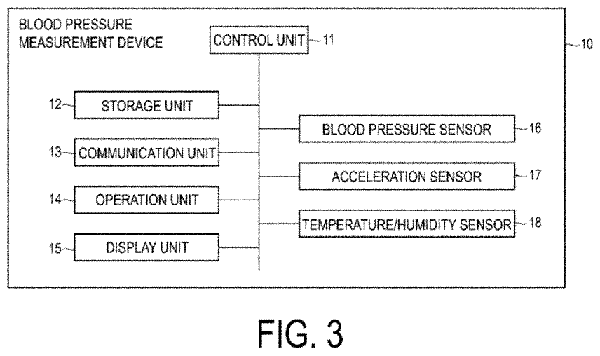

First, a hardware configuration of the blood pressure measurement device 10 according to the present embodiment is described. FIG. 3 is a block diagram illustrating an example of the hardware configuration of the blood pressure measurement device 10 according to the present embodiment. As illustrated in FIG. 3, the blood pressure measurement device 10 according to the present embodiment includes a control unit 11, a storage unit 12, a communication unit 13, an operation unit 14, a display unit 15, a blood pressure sensor 16, an acceleration sensor 17, and a temperature/humidity sensor 18.

The control unit 11 includes a central processing unit (CPU), a random access memory (RAM), a read-only memory (ROM), and the like, and controls the constituent elements in accordance with information processing. Further, the control unit 11 includes a clock (not shown), and has a clock function of displaying current date/time.

The control unit 11 generates blood pressure information, activity information, and environment information based on measurement results obtained by the blood pressure sensor 16, the acceleration sensor 17, and the temperature/humidity sensor 18. Specifically, for example, the blood pressure information includes the blood pressure value of the subject based on measurement performed by the blood pressure sensor 16. The activity information includes an activity amount, the number of steps, and a sleep condition of the subject based on measurement performed by the acceleration sensor 17. The environment information includes temperature and humidity in the periphery of the subject based on measurement performed by the temperature/humidity sensor 18. Each of the blood pressure information, the activity information, and the environment information is associated with the measurement date/time given by the clock. Further, each of the blood pressure information, and the activity information, and the environment information may further be associated with a device ID for uniquely identifying the blood pressure measurement device 10.

For example, the storage unit 12 is an auxiliary storage device such as a hard disk drive (HDD) and a solid state drive (SSD). Until the blood pressure information, the activity information, the environment information, and the like are transmitted to the portable terminal 30, the storage unit 12 temporarily stores those pieces of information.

The communication unit 13 is a communication interface for performing communication with the portable terminal 30. For example, the communication unit 13 transmits, to the portable terminal 30, the blood pressure information, the activity information, the environment information, and the like. For example, in the present embodiment, communication with the portable terminal 30 by the communication unit 13 may adopt near-field wireless communication such as Bluetooth (registered trademark), but is not limited thereto. For example, communication performed by the communication unit 13 may adopt communication via the network NW such as a local area network (LAN) or wired communication through use of a communication cable.

For example, the operation unit 14 includes a user interface such as a touch panel and an operation button. The operation unit 14 detects an operation, which is performed by the subject with the user interface, and outputs a signal indicating a content of the operation to the control unit 11.

For example, the display unit 15 includes a display screen (for example, a liquid crystal display (LCD), an electroluminescence (EL) display, or the like), an indicator, and the like. The display unit 15 displays information in accordance with a signal from the control unit 11, and notifies the subject of the information.

The blood pressure sensor 16 measures a blood pressure value of the subject. For example, the blood pressure value includes a representative index such as a maximum blood pressure and a minimum blood pressure. In the following description, as an example, a case where the blood pressure value is the maximum blood pressure is given. However, the minimum blood pressure and other indexes may be used in place of the maximum blood pressure, and the plurality of indexes may be used in combination.

For example, the blood pressure sensor 16 may be a continuous measurement type capable of measuring blood pressure of the subject at each heartbeat (continuously), or may be a non-continuous measurement type capable of performing measurement at a spot (non-continuously) during a predetermined period. For example, the continuous measurement type blood pressure sensor 16 may adopt a method of measuring blood pressure of the subject continuously based on pulse transit time (PTT), a method of measuring blood pressure of the subject continuously based on a pressure pulse wave (tonometry method), and the like. Note that the method of measuring blood pressure continuously is not limited to the above-mentioned examples, and a method of detecting a pulse wave through use of a light emitting element and the like may be adopted as appropriate. For example, the non-continuous measurement type blood pressure sensor 16 may adopt a method of detecting a pulse wave by applying a pressure on a blood vessel through use of a cuff as a pressure sensor (oscillometric method).

The acceleration sensor 17 detects acceleration of a part of the subject wearing the blood pressure measurement device 10, as a group of three-axial components. Further, the acceleration sensor 17 may further includes a gyro sensor, and may further detect an angular velocity as a group of three-axial components in addition to acceleration.

The temperature/humidity sensor 18 measures temperature and humidity in the periphery of the subject.

### 2.2.2 Hardware Configuration of Portable Terminal

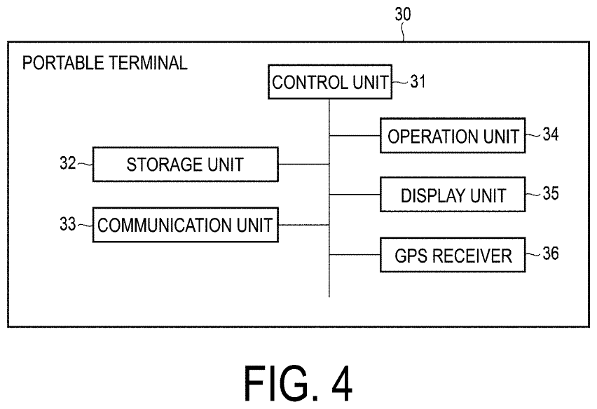

Next, a hardware configuration example of the portable terminal 30 is described. FIG. 4 is a block diagram illustrating an example of the hardware configuration of the portable terminal 30 according to the present embodiment. As illustrated in FIG. 4, the portable terminal 30 according to the present embodiment includes a control unit 31, a storage unit 32, a communication unit 33, an operation unit 34, a display unit 35, and a global positioning system (GPS) receiver 36.

The control unit 31 and the storage unit 32 are similar to the control unit 11 and the storage unit 12 of the blood pressure measurement device 10, respectively. However, until the blood pressure information, the activity information, and the environment information, which are received from the blood pressure measurement device 10, position information generated by the GPS receiver 36, and the like are transmitted to the blood pressure processing device 50, the storage unit 32 of the portable terminal 30 temporarily stores those pieces of information.

The communication unit 33 is a communication interface for performing communication with the blood pressure measurement device 10, the blood pressure processing device 50, and the blood pressure evaluation device 70. For example, the communication unit 33 receives the blood pressure information, the activity information, the environment information, and the like from the blood pressure measurement device 10. Further, the communication unit 33 transmits the blood pressure information, the activity information, the environment information, the position information, and the like to the blood pressure processing device 50, and receives the evaluation information from the blood pressure evaluation device 70. For example, in the present embodiment, communication by the communication unit 33 with the blood pressure processing device 50 and the blood pressure evaluation device 70 may adopt communication via the network NW, but is not limited thereto. Near-field wireless communication or wired communication may be adopted.

The operation unit 34 and the display unit 35 are similar to the operation unit 14 and the display unit 15 of the blood pressure measurement device 10, respectively.

The GPS receiver 36 measures a position of the portable terminal 30, and generates the position information. For example, the position information includes a positioning date/time and a latitude and a longitude of the portable terminal 30 at the positioning date/time. For example, positioning performed by the GPS receiver 36 may be performed in synchronization with measurement performed by the blood pressure sensor 16 of the blood pressure measurement device 10.

### 2.2.3 Hardware Configuration of Blood Pressure Processing Device

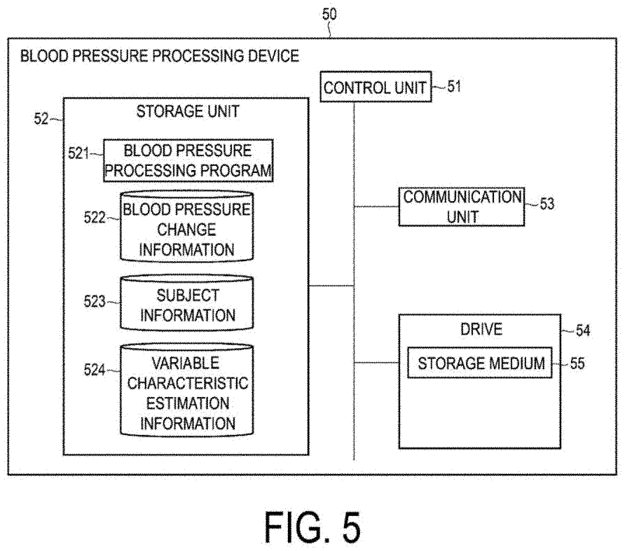

Next, a hardware configuration example of the blood pressure processing device 50 is described. FIG. 5 is a block diagram illustrating an example of the hardware configuration of the blood pressure processing device 50 according to the present embodiment. As illustrated in FIG. 5, the blood pressure processing device 50 according to the present embodiment includes a control unit 51, a storage unit 52, a communication unit 53, and a drive 54.

The control unit 51 and the storage unit 52 are similar to the control unit 11 and the storage unit 12 of the blood pressure measurement device 10, respectively. However, the storage unit 52 of the blood pressure processing device 50 stores a blood pressure processing program 521 executed by the control unit 51 and blood pressure change information 522, subject information 523, variable characteristic estimation information 524, and the like, which are used in the blood pressure processing program 521.

The communication unit 53 is a communication interface for performing communication with the portable terminal 30 and the blood pressure evaluation device 70. For example, the communication unit 53 receives the blood pressure information, the activity information, the environment information, the position information, and the like from the portable terminal 30. Further, the communication unit 53 transmits the analysis information on the blood pressure value to the blood pressure evaluation device 70.

For example, the drive 54 is a compact disk (CD) drive, a digital versatile disk (DVD) drive, or the like, and is a device for reading a program stored in a storage medium 55. A type of the drive 54 may be selected as appropriate in accordance with a type of the storage medium 55. The blood pressure processing program 521, and the blood pressure change information 522, the subject information 523, and the variable characteristic estimation information 524 may be stored in the storage medium 55.

The storage medium 55 is a medium for accumulating information such as the program electrically, magnetically, optically, mechanically, or chemically so that a computer and other device or machine may read the stored information such as the program. The blood pressure processing device 50 may acquire, from the storage medium 55, the blood pressure processing program 521 and the blood pressure change information 522, the subject information 523, and the variable characteristic estimation information 524.

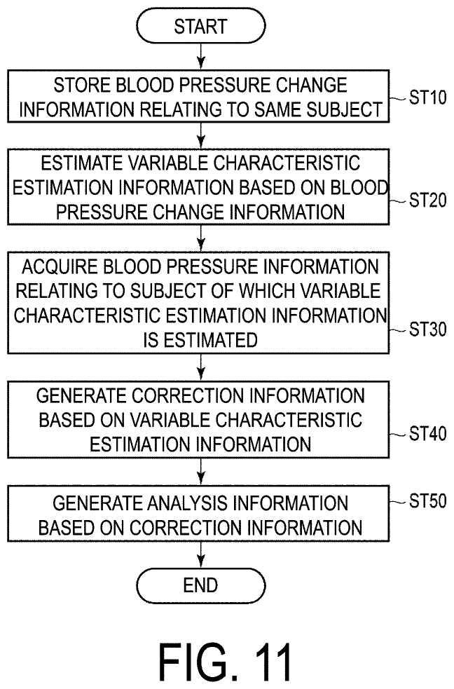

The blood pressure processing program 521 is a program for causing the blood pressure processing device 50 to execute blood pressure analysis processing for subjecting a blood pressure value to analysis processing, which is described later (FIG. 11).

The blood pressure change information 522 is information obtained by integrating the plurality pieces of blood pressure information relating to the same subject. The plurality pieces of blood pressure information include blood pressure values measured at different measurement times within the period. With this, the blood pressure values of the subject, which correspond to the blood pressure change information 522, can be grasped discretely over the entire period.

The subject information 523 is information relating to the subject having a blood pressure value being an evaluation target. For example, in the subject information 523, personal information including, a name, an age, a sex, an address, an obesity degree, presence or absence of a smoking habit, and the like and a device ID of the blood pressure measurement device 10 that is carried by the subject corresponding to the personal information are associated with each other, and the subject information 523 is stored in the storage unit 52. For example, the obesity degree includes a body fat percentage, a body mass index (BMI), and the like. The subject information 523 is updated as appropriate by a statement made by the subject.

The variable characteristic estimation information 524 is information indicating a state in which a blood pressure value cyclically varies during a period having a predetermined length as a cycle. Further, the variable characteristic estimation information 524 is estimated based on the blood pressure change information 522 relating to the same subject. Specifically, over the entire period, the variable characteristic estimation information 524 continuously indicates the variable characteristics of the blood pressure of the subject, which is associated with the blood pressure change information 522, and the most probable variation amount of each time within the cycle.

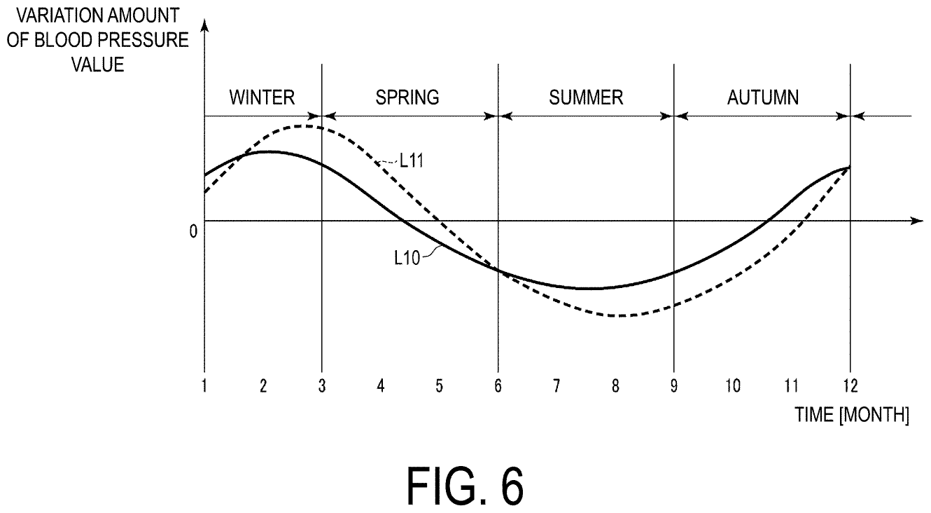

FIG. 6 is a diagram illustrating an example of the variable characteristic estimation information 524 according to the present embodiment. In the example in FIG. 6, the variable characteristic estimation information 524 indicates a state in which a blood pressure cyclically varies about a predetermined average value for a year as a cycle. Further, in the example in FIG. 6, two variation curves L10 and L11, which are estimated for different subjects, are shown. Note that, in the example in FIG. 6, it is assumed that both the variation curves L10 and L11 indicate variable characteristics of the subjects in the same region in the same country (for example, in Japan).

As illustrated in FIG. 6, when a year is set as a cycle of blood pressure variation, the variation curves L10 and L11 each indicate a variation amount of a blood pressure value that rises and falls with respect to an average value (for example, “0”) depending on a month (or a season). More specifically, for example, the variation curves L10 and L11 each indicate a variation amount of a blood pressure value that substantially rises with respect to the average value (the variation amount “0” of the blood pressure value) during a period from December to March (winter) and substantially falls with respect to the average value during a period from June to September (summer). Further, for example, the variation amount of the blood pressure value evolves near the average value during a period from March to June (spring) and a period from September to December (autumn). The variation amplitude of those variation curves L10 and L11 falls within a range of from approximately 10 mmHG to 20 mmHG at most.

However, as described above, the variable characteristic estimation information 524 is unique to each subject, and hence the variable characteristics indicated with the variation curves L10 and L11 do not necessarily match with each other. For example, in the example in FIG. 6, the variation curve L11 has an amplitude of the variation amount larger than that of the variation curve L10, and has belated periods during which the variation amount is the maximum and minimum, approximately by a month as compared to the variation curve L10.

Note that FIG. 6 is merely an example, the variable characteristics indicated with the variation curves L10 and L11 are not limited to the variable characteristics and the variation amounts illustrated in FIG. 6, and may be freely-selected variable characteristics and variation amounts that are different from the trends in the example in FIG. 6. For example, changing of the seasons caused by the rotation of the earth is conceived as one of the factors that cause one-year cyclic variation of blood pressure. Thus, as described above, FIG. 6 shows the variable characteristics of the blood pressure values acquired from the subjects living in Japan. However, variable characteristics of a blood pressure value acquired from a subject living in a country or a region having seasonal change and a climate that are different from those in Japan may be different from the variable characteristics shown in FIG. 6. Specifically, depending on a measurement location of a blood pressure value (a difference in a country and a region or a difference in a latitude and a longitude), variable characteristics may vary. Note that, in the following description, description is made with reference to variable characteristics obtained from a subject living in the same country and region as in the variable characteristics shown in FIG. 6.

### 2.2.4 Hardware Configuration of Blood Pressure Evaluation Device

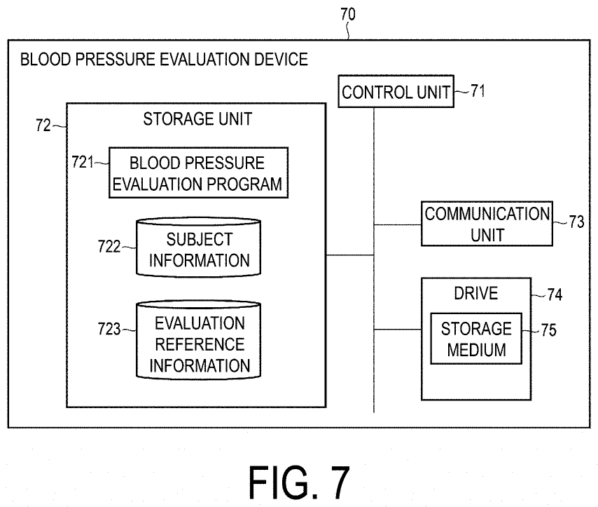

Next, a hardware configuration example of the blood pressure evaluation device 70 is described. FIG. 7 is a block diagram illustrating an example of a hardware configuration of the blood pressure evaluation device 70 according to the present embodiment. As illustrated in FIG. 7, the blood pressure evaluation device 70 according to the present embodiment includes a control unit 71, a storage unit 72, a communication unit 73, and a drive 74.

The control unit 71 and the storage unit 72 are similar to the control unit 11 and the storage unit 12 of the blood pressure measurement device 10, respectively. However, the storage unit 72 of the blood pressure evaluation device 70 stores a blood pressure evaluation program 721 executed by the control unit 71 and subject information 722 and evaluation reference information 723, which are used in the blood pressure evaluation program 721.

The communication unit 73 is a communication interface for performing communication with the portable terminal 30 and the blood pressure processing device 50. For example, the communication unit 73 receives the analysis information and the like from the blood pressure processing device 50. Further, the communication unit 73 transmits the evaluation information relating to the blood pressure value to the portable terminal 30.

The drive 74 and a storage medium 75 are similar to the drive 54 and the storage medium 55 of the blood pressure processing device 50, respectively. However, the blood pressure evaluation program 721, and the subject information 722 and the evaluation reference information 723 may be stored in the storage medium 75. Specifically, the blood pressure evaluation device 70 may acquire, from the storage medium 75, the blood pressure evaluation program 721, as well as the subject information 722 and the evaluation reference information 723.

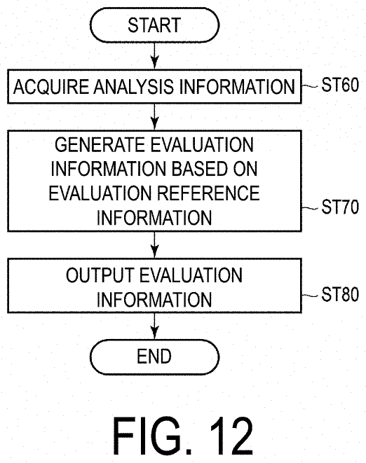

The blood pressure evaluation program 721 is a program for causing the blood pressure evaluation device 70 to execute blood pressure evaluation processing described later (FIG. 12).

The subject information 722 is similar to the subject information 523 of the blood pressure processing device 50.

The evaluation reference information 723 is information for defining a relationship between a blood pressure value and an evaluation result. The evaluation reference information 723 may adopt various modes in accordance with evaluation contents. When an evaluation content is information indicating whether the subject is allowed to purchase insurance, for example, the evaluation reference information 723 sets a blood pressure value as a threshold value, and associates a blood pressure value exceeding the threshold value with information indicating a denial of insurance purchase and associates a blood pressure value falling below the threshold value with information indicating an acceptance of insurance purchase. Further, when an evaluation content is insurance premium or reward points of the subject, for example, the evaluation reference information 723 sets an output value of a function including a blood pressure value as an argument, and associates the insurance premium or reward points with the blood pressure value. In the evaluation reference information 723, different references may be set in accordance with attribute information relating to the subject.

### 2. 3 Functional Configuration Examples

Next, an example of a functional configuration of the blood pressure processing system according to the present embodiment is described.

### 2.3.1 Functional Configuration Example of Blood Pressure Evaluation Device

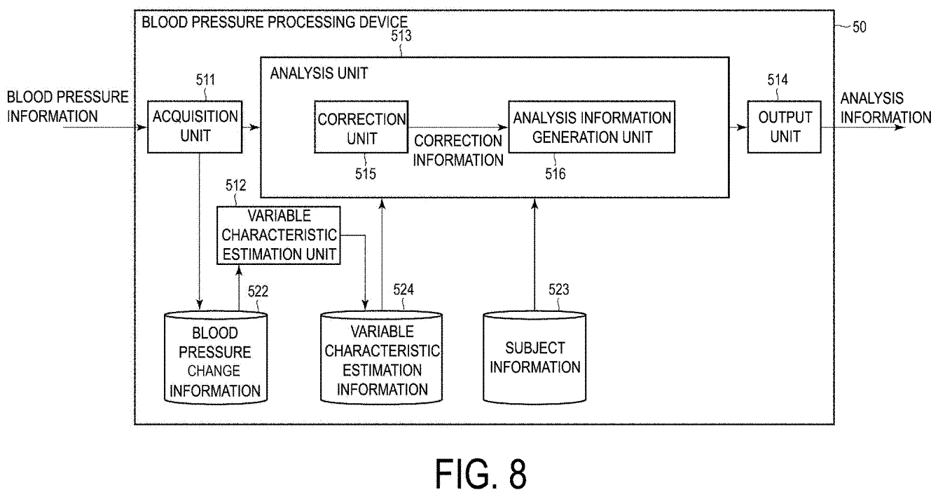

FIG. 8 is a block diagram schematically illustrating an example of a functional configuration of the blood pressure processing device 50 of the blood pressure processing system according to the present embodiment.

The control unit 51 of the blood pressure processing device 50 develops the blood pressure processing program 521, which is stored in the storage unit 52, in the RAM. Further, the control unit 51 reads and executes the blood pressure processing program 521, which is developed in the RAM, with the CPU, and controls the constituent elements. With this, as illustrated in FIG. 8, the blood pressure processing device 50 according to the present embodiment functions as a computer including an acquisition unit 511, a variable characteristic estimation unit 512, an analysis unit 513, and an output unit 514.

The acquisition unit 511 acquires various types of information measured by the blood pressure measurement device 10 and the portable terminal 30. Specifically, for example, the acquisition unit 511 acquires the blood pressure information, and forwards the information to the storage unit 52 and the analysis unit 513. The blood pressure information forwarded to the storage unit 52 is collected for each subject, and is stored as the blood pressure change information 522.

Based on the blood pressure change information 522, the variable characteristic estimation unit 512 estimates the variable characteristic estimation information 524 for the subject corresponding to the blood pressure change information 522. Estimation of the variable characteristic estimation information 524, which is performed by the variable characteristic estimation unit 512, is described later.

The analysis unit 513 corrects a cyclic variation amount immanent in a blood pressure value, and corrects and analyzes the blood pressure value. Specifically, the analysis unit 513 includes a correction unit 515 and an analysis information generation unit 516.

When receiving the blood pressure information from the acquisition unit 511, the correction unit 515 extracts the measurement date/time of the blood pressure information. The correction unit 515 reads the variable characteristic estimation information 524, and specifies a time within the cycle, which corresponds to the measurement date/time extracted from the blood pressure information. The correction unit 515 extracts, from the variable characteristic estimation information 524, a variation amount of the blood pressure value, which corresponds to the specified time within the cycle, and corrects the blood pressure value based on the variation amount. Specifically, for example, the correction unit 515 subtracts the cyclic variation amount from the blood pressure value, and calculates a corrected blood pressure value that is independent of the measurement date/time. More specifically, the corrected blood pressure value may be calculated as an average value of the blood pressure value varying during the period. The correction unit 515 rewrites the blood pressure value in the blood pressure information into the corrected blood pressure value, and generates correction information. The correction unit 515 transmits the generated correction information to the analysis information generation unit 516.

When receiving the correction information, the analysis information generation unit 516 generates the analysis information through use of the variable characteristic estimation information 524. The analysis information includes information that may be used for evaluating the blood pressure value, together with the correction information. When evaluation is performed by the blood pressure evaluation device 70, which is described later, combination of the analysis information with the corrected blood pressure value can improve accuracy of the evaluation information. Specifically, for example, the analysis information includes a variation amplitude, a maximum value, a minimum value and a most frequent value of the blood pressure value within the period in addition to the corrected blood pressure value. With this, the information indicating a degree at which the blood pressure value of the subject varies with respect to the corrected blood pressure value as an average can be taken into consideration at the time of evaluation. The analysis information generation unit 516 transmits the generated analysis information to the output unit 514.

The output unit 514 outputs the evaluation information to the blood pressure evaluation device 70.

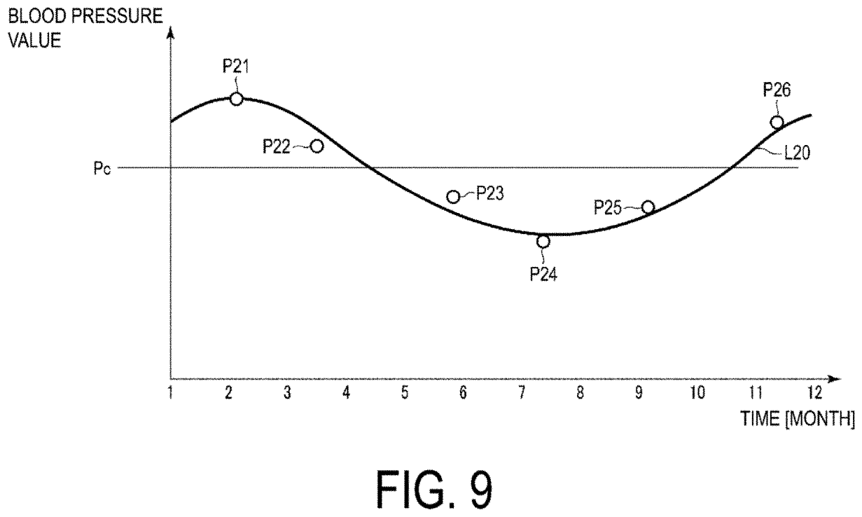

FIG. 9 is a diagram illustrating an example of a relationship between the blood pressure change information 522 and the variable characteristic estimation information 524 according to the present embodiment. In the example in FIG. 9, the blood pressure change information 522 includes blood pressure information pieces P21, P22, P23, P24, P25, and P26 measured from the same subject at six measurement times. Further, a variation curve L20 indicates the variable characteristic estimation information 524 estimated from the blood pressure change information 522. It is desired that the variation curve L20 allows accurate approximation of the blood pressure variation of the subject over the entire period.

For example, as illustrated in FIG. 9, the variable characteristic estimation unit 512 estimates the variation curve L20 so that an error from each of the blood pressure information pieces P21 to P26 is minimized. For example, the estimation method may adopt a least squares method and the like as appropriate, but is not limited thereto. A freely selected maximum likelihood estimation is adoptable. Specifically, the variable characteristic estimation unit 512 may estimate the variation curve L20 while weighting each of the blood pressure information pieces P21 to P26. Specifically, for example, among the blood pressure information pieces P21 to P26, the variable characteristic estimation unit 512 may apply higher weighting to an information piece measured at a measurement time that is different from those of the other blood pressure information pieces, and may apply lower weighting to the plurality pieces of blood pressure information, which are measured at a substantially same measurement time.

Further, the variable characteristic estimation unit 512 may express the estimated variable characteristics of the variation curve L20 in a formula in a freely-selected form such as a polynomial expression and a trigonometric function. With this, based on the variable characteristic estimation information 524, the analysis unit 513 can estimate a variation amount at a measurement time within the period, which is not directly included in the blood pressure change information 522. Note that the variation curve L20 may be expressed in a formula so that a variation amount from an average value Pc is an output value. With this, the variation curve L20 can be applied by being shifted upward and downward in a blood pressure value direction regardless of the value of the average value Pc.

### 2.3.2 Functional Configuration Example of Blood Pressure Evaluation Device

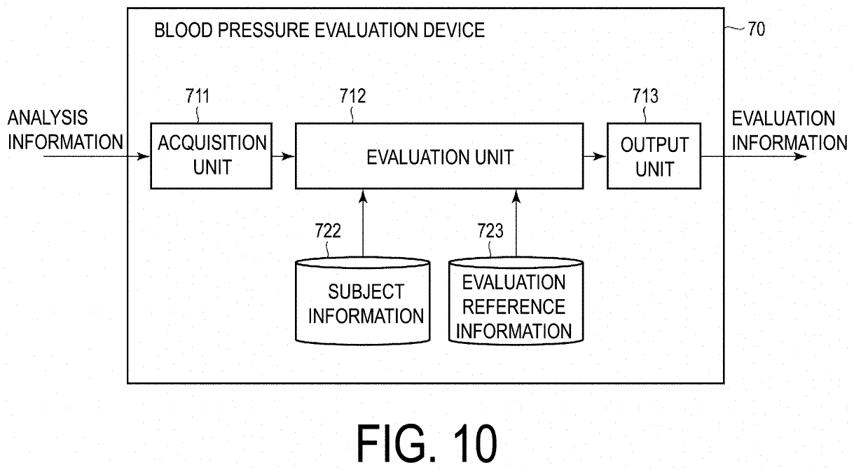

FIG. 10 is a block diagram schematically illustrating an example of a functional configuration of the blood pressure evaluation device 70 of the blood pressure processing system according to the present embodiment.

The control unit 71 of the blood pressure evaluation device 70 develops the blood pressure evaluation program 721, which is stored in the storage unit 72, in the RAM. Further, the control unit 71 reads and executes the blood pressure evaluation program 721, which is developed in the RAM, with the CPU, and controls the constituent elements. With this, as illustrated in FIG. 10, the blood pressure evaluation device 70 according to the present embodiment functions as a computer including an acquisition unit 711, an evaluation unit 712, and an output unit 713.

The acquisition unit 711 acquires the analysis information output from the blood pressure processing device 50.

When receiving the analysis information, the evaluation unit 712 reads the subject information 722, and specifies an individual associated with the device ID in the analysis information. The evaluation unit 712 sets the evaluation reference information 723 corresponding to attribute information relating to the specified individual, and generates the evaluation information corresponding to the corrected blood pressure value. Further, the evaluation unit 712 may generate the evaluation information in consideration of information such as a variation amplitude of the blood pressure value together with the corrected blood pressure value.

For example, as the evaluation result, the evaluation information may include information for determining whether the subject is allowed to purchase desired insurance and information for calculating an insurance premium of health insurance that the subject purchases or reward points such as a coupon that is given in accordance with a health state of the subject.

The output unit 713 outputs the evaluation information to the portable terminal 30.

### 2.4 Operation Examples

Next, an operation example of the blood pressure processing system according to the present embodiment is described. Note that a processing procedure described below is merely an example, and each processing may be changed as much as possible. Further, omission, replacement, and addition of steps may be made to the processing procedure described below as appropriate in accordance with embodiments.

### 2.4.1 Operation Example of Blood Pressure Processing Device

FIG. 11 is a flowchart schematically illustrating an example of a procedure of blood pressure analysis processing in the blood pressure processing device 50 according to the present embodiment.

### Step ST10

First, in Step ST10, the control unit 51 causes the storage unit 52 to store the plurality pieces of blood pressure information relating to the same subject as the blood pressure change information 522. Note that the subject can be specified by the subject information 523.

### Step ST20

In Step ST20, the control unit 51 functions as the variable characteristic estimation unit 512, and estimates the variable characteristic estimation information 524 based on the blood pressure change information 522 stored in Step ST10.

### Step ST30

In Step ST30, the control unit 51 causes the communication unit 53 to function as the acquisition unit 511, and acquires the blood pressure information, which is measured by the blood pressure measurement device 10, via the portable terminal 30. The measurement time of the blood pressure information to be acquired may be different from the measurement time of the blood pressure information in the blood pressure change information 522 stored in Step ST10. Further, the communication unit 53 may further acquire the activity information, the environment information and the position information.

### Step ST40

In Step ST40, the control unit 51 functions as the correction unit 515, and generates the correction information for the blood pressure information acquired in Step ST30, based on the variable characteristic estimation information 524 stored in Step ST20.

Specifically, the control unit 51 extracts, from the variable characteristic estimation information 524, the variation amount of the blood pressure value corresponding to the measurement date/time in the blood pressure information. The control unit 51 subtracts the extracted variation amount from the blood pressure value, and calculates the corrected blood pressure value that is independent of the cyclic variation. The control unit 51 generates the correction information including the corrected blood pressure value, and transmits the correction information to the analysis information generation unit 516.

### Step ST50

In Step ST50, the control unit 51 functions as the analysis information generation unit 516, and generates the analysis information based on the correction information generated in Step ST40.

Specifically, the control unit 51 calculates the variation amplitude and the like of the blood pressure value about the corrected blood pressure value based on the variable characteristic estimation information 524, adds the calculated result to the correction information, and thus generates the analysis information.

### Output

Finally, the control unit 51 causes the communication unit 53 to function as the output unit 514, and outputs the analysis information generated in Step ST50 to the blood pressure evaluation device 70.

Thus, the operation of the blood pressure processing device 50 is completed.

### 2.4.2 Operation Example of Blood Pressure Evaluation Device

FIG. 12 is a flowchart schematically illustrating an example of a procedure of the blood pressure evaluation processing in the blood pressure evaluation device 70 according to the present embodiment.

### Step ST60

In Step ST60, the control unit 71 causes the communication unit 73 to function as the acquisition unit 711, and acquires the analysis information from the blood pressure processing device 50.

### Step ST70

In Step ST70, the control unit 71 functions as the evaluation unit 712, evaluates the analysis information acquired in Step ST50 based on the subject information 722 and the evaluation reference information 723, and generates the evaluation information.

Specifically, with the device ID in the analysis information, the control unit 71 specifies the attribution information in the subject information 722 corresponding to the blood pressure value in the blood pressure information. Further, the control unit 71 sets the evaluation reference information 723 in accordance with the specified attribution information. The control unit 71 evaluates the corrected blood pressure value and other information in the analysis information based on the set evaluation reference information 723, and generates the evaluation information including the evaluation result.

### Step ST80

In Step ST80, the control unit 71 causes the communication unit 73 to function as the output unit 713, and outputs the evaluation information generated in Step ST70 to the portable terminal 30. For example, the subject is notified of the output evaluation information with the display unit 35 of the portable terminal 30.

Thus, the operation of the blood pressure evaluation device 70 is completed.

### 2.5 Actions and Effects

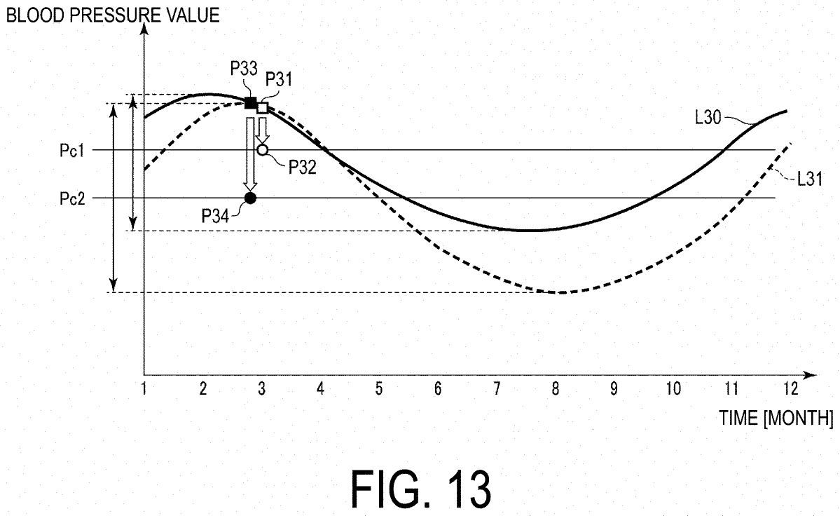

According to the present embodiment, the technology that enables the blood pressure value of the subject to be processed appropriately and independently of the subject can be provided. The present effects are described below with reference to FIG. 13. FIG. 13 is a diagram illustrating an example of the effects of the present embodiment.

The example in FIG. 13 shows a case where two pieces of variable characteristic estimation information 524 having different variable characteristics are estimated from different subjects in Step ST20. The two pieces of variable characteristic estimation information 524 are indicated with variation curves L30 and L31, respectively. Specifically, the subject corresponding to the variation curve L30 has a variation amplitude of blood pressure, which is larger than that of the subject corresponding to the variation curve L31.

Further, the example in FIG. 13 shows a case where a blood pressure information P31 is acquired from the subject corresponding to the variation curve L30 and a blood pressure information P33 is acquired from the subject corresponding to the variation curve L31 in Step ST30. The measurement times of blood pressure information P31 and P33 are the same, and the blood pressure values are also at the same degree.

In this case, in Step ST40, the control unit 51 applies the variation curves L30 and L31 to the blood pressure information P31 and P33, respectively, and performs correction to correction information P32 and P34 including the corrected blood pressure values. The variation curves L30 and L31 have the variation amplitudes that are different from each other, and hence the corrected blood pressure values of the correction information P32 and P34 are values that are different from each other. In the example in FIG. 13, a corrected blood pressure value Pc1 in the correction information P32 is larger than a corrected blood pressure value Pc2 in the correction information P34.

As described above, according to the present embodiment, when the blood pressure values at the same degree are measured at the same time from the two subjects having the variable characteristics that are different from each other, the blood pressure values can be corrected in accordance with the subjects. Thus, in Step ST50, the control unit 51 can generate the corrected blood pressure values Pc1 and Pc2 from the two pieces of blood pressure information P31 and P33. Therefore, the blood pressure processing device 50 can provide the information, which can perform evaluation that the blood pressure information P31 and P33 have different risks, to the blood pressure evaluation device 70.

Further, in Step ST50, the control unit 51 can include the information, which indicates a degree at which the blood pressure values of the subjects vary with respect to the corrected blood pressure values Pc1 and Pc2 as the average values, in the analysis information, in addition to the corrected blood pressure values Pc1 and Pc2. With this, the blood pressure processing device 50 can provide the information for evaluating the risks of the subjects more accurately to the blood pressure evaluation device 70.

### 3. Modified Examples

The detailed description is made above on the embodiment of the present invention, and is merely an example of the present invention in every aspect of the above-given description. It is needless to say that various improvements and modifications can be made without departing the gist of the present invention. For example, modifications described below can be made. Note that, in the following description, the same constituent elements as those in the embodiment are denoted with the same reference symbols, and the same matters as those in the embodiment are omitted in description as appropriate. The modified examples described below may be combined as appropriate.

### 3.1 First Modified Example

For example, in the embodiment, the case where the cycle of the blood pressure variation in the variable characteristic estimation information 524 is one year is described. The cycle of the blood pressure variation is not limited to one year but may adopt a freely-selected period as appropriate as long as the blood pressure variation is statistically cyclic during the period.

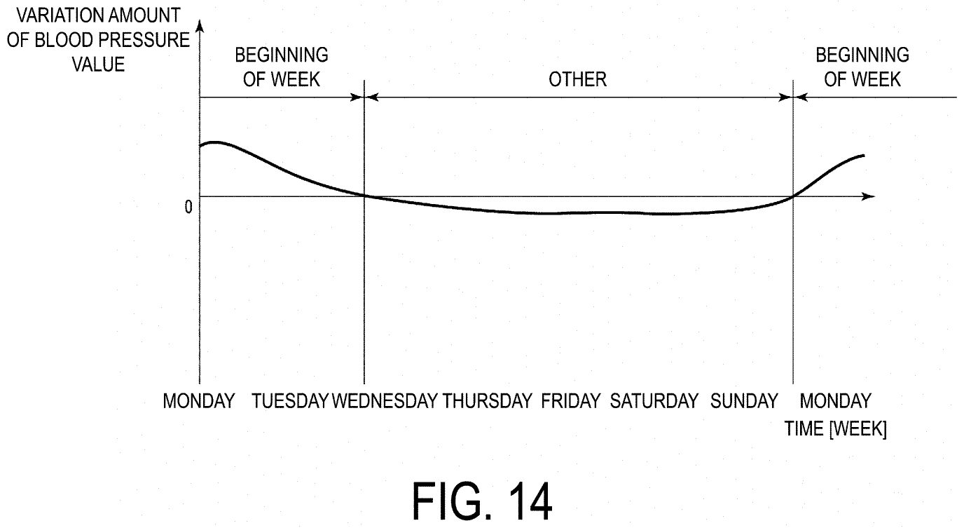

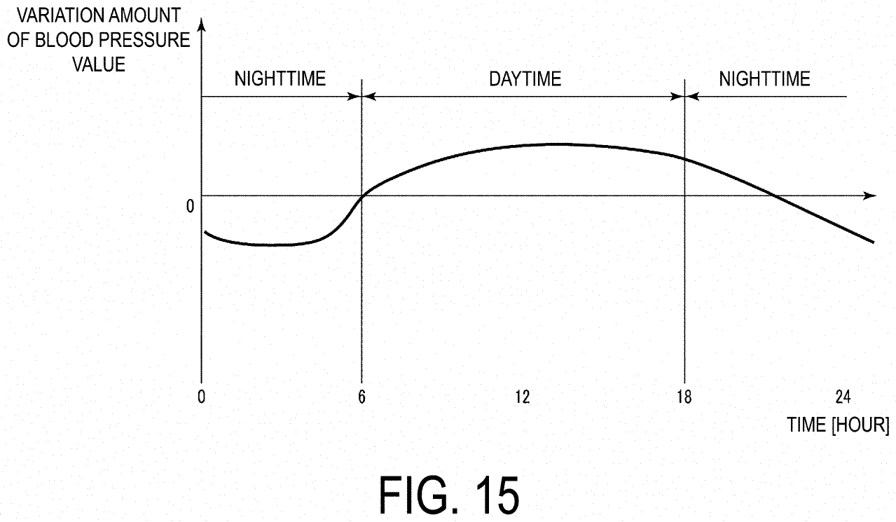

Specifically, for example, the cycle of the blood pressure variation may be set to either a week or a day. FIG. 14 and FIG. 15 are diagrams illustrating examples of the variable characteristic estimation information 524 according to the present embodiment in a first modified example. The variable characteristic estimation information 524 indicates a state in which the blood pressure value cyclically varies for a week and a day as a cycle in the examples in FIG. 14 and FIG. 15, respectively. Note that, in the examples in FIG. 14 and FIG. 15, each of the variation curves indicates variable characteristics relating to a subject who works in the daytime on weekdays and rests in the nighttime on weekdays, and Saturdays and Sundays.

As illustrated in FIG. 14, when the cycle of the blood pressure variation is set to a week, a variation amount of the blood pressure value rises and falls with respect to an average value (for example, “0”) depending on a day. More specifically, for example, the variation amount of the blood pressure value rises with respect to the average value during a period from Monday to Tuesday (the beginning of the week), and falls with respect to the average value during a period from Wednesday to Sunday (others).

Further, as illustrated in FIG. 15, when the cycle of the blood pressure variation is set to a day, a variation amount of the blood pressure value rises and falls with respect to an average value (for example, “0”) depending on the time. More specifically, for example, the variation amount of the blood pressure value rises with respect to the average value during a period (daytime) from daybreak (for example, 6:00 am) to sundown (for example, 6:00 pm), and falls with respect to the average value during a period (nighttime) from sundown to daybreak.

As described above, in general, various cyclically variable components may overlap in the blood pressure value acquired from the subject. Note that FIG. 14 and FIG. 15 are merely examples, and in addition to the variable characteristics and the variation amounts illustrated in FIG. 14 and FIG. 15, freely-selected variable characteristics and variation amounts, which have tendencies different from those in the examples in FIG. 14 and FIG. 15, may overlap in the variable characteristic estimation information 524. For example, as one of the factors that cause one-week cyclic variation of blood pressure, a one-week cycle including working days and rest days is possible. Thus, as described above, FIG. 14 shows the variable characteristics of the blood pressure value acquired from the subject who works on weekdays and rests on Saturdays and Sundays, but variable characteristics of a blood pressure value acquired from a subject who works on Saturdays or Sundays may be different from the variable characteristics shown in FIG. 14. Further, for example, as one of the factors that cause one-day cyclic variation of blood pressure, a one-day cycle including working hours and rest hours is possible. Thus, as described above, FIG. 15 shows the variable characteristics of the blood pressure value acquired from the subject who works in the daytime and takes a rest in the nighttime, but variable characteristics of a blood pressure value acquired from a subject who works in the nighttime may be different from the variable characteristics shown in FIG. 15. Further, for example, variable characteristics of a blood pressure value acquired from a jobless subject (for example, a subject who does not work at no point in time on any day) may be different from the variable characteristics shown in FIG. 14 and FIG. 15.

Based on the blood pressure change information 522, the variable characteristic estimation unit 512 may estimate the variable characteristic estimation information 524 for each of a plurality of different cycles. For example, the variable characteristic estimation unit 512 may estimate the plurality pieces of variable characteristic estimation information 524 by filtering the plurality pieces of blood pressure information in the blood pressure change information 522. Specifically, for example, the variable characteristic estimation unit 512 may estimate the variable characteristic estimation information 524 for a plurality of different cycles by subjecting the plurality pieces of blood pressure information to Fourier transform for frequency conversion and extracting frequency components having a desired unit period as a cycle.

Further, the correction unit 515 may sequentially correct a blood pressure based on each of the plurality pieces of variable characteristic estimation information 524 estimated for the different cycles.

With this, various factors for cyclic blood pressure variation can be taken into consideration, and the blood pressure value of the subject can be processed more appropriately.

### 3.2 Second Modified Example

Further, in the embodiment, the case where the blood pressure information used for generation of the evaluation information is not used for estimation of the variable characteristic estimation information 524 is described, but the embodiment is not limited thereto. For example, the variable characteristic estimation information 524 may be estimated from the blood pressure change information 522 in which the latest blood pressure information is reflected. Further, for example, the blood pressure change information 522 may dispose blood pressure information for a predetermined period (for example, one year) before acquisition of the latest blood pressure information. With this, the variable characteristic estimation information 524 can be updated with the latest blood pressure information. Thus, when the variable characteristics of the subject varies with the lapse of time, the impact can be reduced.

Further, for example, when the subject starts or quits smoking, or moves to a region having a different climate (for example, migration from a region with extreme temperature differences to a region with mild temperature differences and the like, it is possible that the variable characteristics of the blood pressure values of the subject vary non-continuously. Thus, when there is change in a state of a smoking habit or an address at the time of updating the subject information 523, and the position information from the portable terminal 30 indicates a location different from the past, the blood pressure change information 522 may be newly collected. With this, an impact of the change in variable characteristics of the blood pressure value of the subject along with change of living environment, a lifestyle, and the like of the subject can be taken into consideration.

### 3.3 Third Modified Example

Further, in the embodiment, the case where only the blood pressure information acquired from the specified subject is used for correction, but the embodiment is not limited thereto. For example, the variable characteristic estimation information 524 may be estimated by combining the blood pressure change information 522 with statistic information on variable characteristics of biological information relating to at least one unspecified subject during a unit period including a plurality of times.

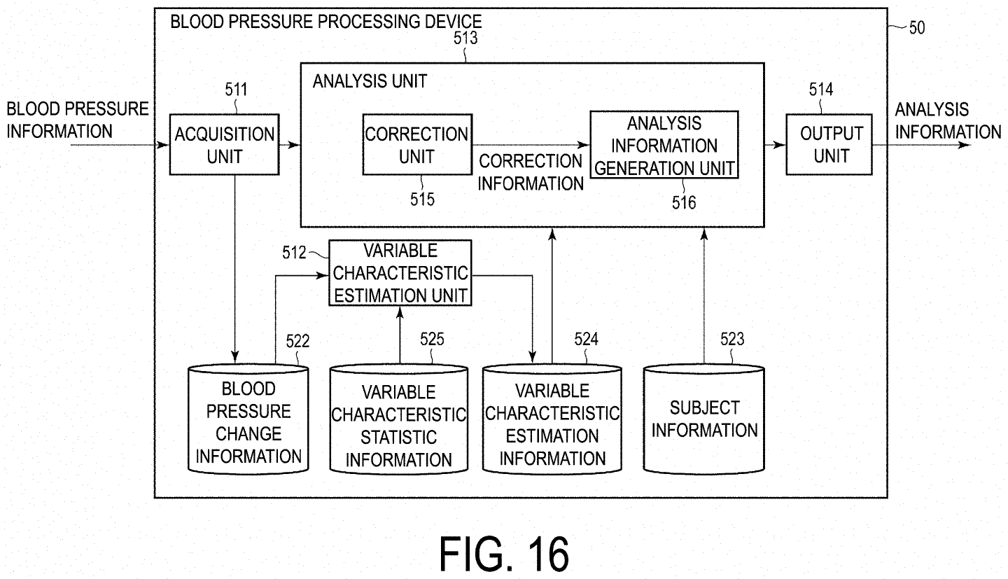

FIG. 16 is a block diagram illustrating a functional configuration of the blood pressure processing device 50 according to the present embodiment in a third modified example. As illustrated in FIG. 16, the variable characteristic estimation unit 512 estimates the variable characteristic estimation information 524 based on the blood pressure change information 522 and variable characteristic statistic information 525.

The variable characteristic statistic information 525 is statistically calculated based on blood pressure information acquired from many unspecified subjects other than the specified subject being an evaluation target, and is stored in the storage unit 52 in advance. Specifically, the variable characteristic statistic information 525 indicates a variation amount observed in the variable characteristics of the blood pressure value shared among most of the subjects used for generation of the information, specifically, a variation amount that is most probable at the time within the cycle.

As described above, the variable characteristics of the blood pressure value of the specified subject may be different from the statistic variable characteristics of the blood pressure values of many unspecified subjects. However, it is assumed that even though the variable characteristics of the blood pressure value of the specified subject are different from the statistic variable characteristics for a part of the entire period, the overall variation trends of the both are the same in many cases. Further, a case where a sufficient amount of the blood pressure information, which enables sufficiently accurate estimation of the variable characteristic estimation information 524 cannot be acquired from the specified subject is also assumed.

Thus, when a period during which a blood pressure value is not sufficiently acquired in the blood pressure change information 522 is present, the variable characteristic estimation unit 512 may estimate the variable characteristic estimation information 524 by applying the variable characteristic statistic information 525 during the period.

With this, even in a state in which sufficient blood pressure information is not acquired over an entire period, the variable characteristic estimation information 524 can be estimated without being deviated from the overall variation trend. Therefore, the blood pressure value of the subject can be processed appropriately.

### 3.4 Fourth Modified Example

Further, in the embodiment, the case where the blood pressure processing device 50 and the blood pressure evaluation device 70 are configured as different server devices is described, but the embodiment is not limited thereto. For example, the functional configurations in each of the blood pressure processing device 50 and the blood pressure evaluation device 70 may be configured within one server device. In this case, a target to be corrected by the correction unit 515 may not be limited to a blood pressure value. For example, the correction unit 515 may generate the correction information by correcting the evaluation reference information 723 in accordance with the measurement time in the blood pressure information.

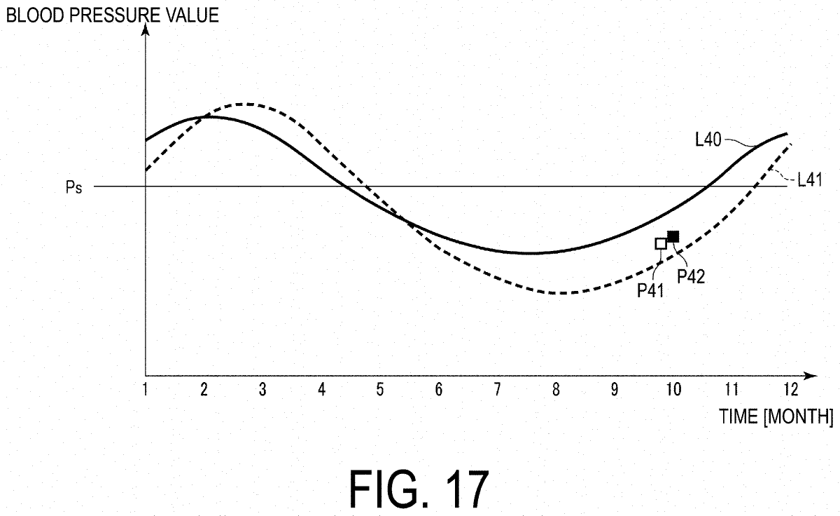

FIG. 17 is a diagram illustrating the evaluation reference information 723 according to the present embodiment in a fourth modified example. In the example in FIG. 17, a case where blood pressure information P41 and P42 are acquired from different subjects is illustrated. Both the blood pressure values in the blood pressure information P41 and P42 are lower than a threshold value Ps independent of a measurement time. In this case, for example, the correction unit 515 may cause the threshold value Ps to vary based on the variable characteristics corresponding to each of the subjects. Specifically, the correction unit 515 may correct the threshold value Ps as a corrected threshold value L40 with respect to a subject of the blood pressure information P41, and may correct the threshold value Ps as a corrected threshold value L41 with respect to a subject of the blood pressure information P42. Note that it is preferred that the corrected threshold values L40 and L41 have a form in a relation with the variable characteristic estimation information 524 relating to the corresponding subjects.

With this, it is understood that the blood pressure value in the blood pressure information P41 is lower than the corrected threshold value L40 whereas the blood pressure value in the blood pressure information P42 exceeds the corrected threshold value L41. In addition, the evaluation unit 712 can evaluate that the blood pressure information P41 and P42 have different risks.

### 3.5 Others

Further, in the embodiment, the case where the variable characteristic estimation information 524 is continuous information over the entire period is described, but the embodiment is not limited thereto. For example, the variable characteristic estimation information 524 may be information that is non-continuous at a certain time within the period as a border. Specifically, for example, the variable characteristic estimation information 524 may be information that can determine a variation amount for each of the four seasons including spring, summer, autumn, and winter when a unit period is set to a year.

Further, in the embodiment, the case where the corrected blood pressure value is an average value of the blood pressure value that cyclically varies is described, but the embodiment is not limited thereto. For example, the corrected blood pressure value may adopt a freely-selected value such as a maximum value, a minimum value, a most frequent value, and other values of the blood pressure value that cyclically varies.

Further, in the embodiment, the description is made by assuming that each of the functional configurations in the blood pressure processing device 50 is included in the server device connected to the portable terminal 30 via the network NW, but the embodiment is not limited thereto. For example, each of the functional configurations in the blood pressure processing device 50 may be included in the blood pressure measurement device 10 and the portable terminal 30.

Further, in the embodiment, the description is made by assuming that the blood pressure measurement device 10 is a device that can be worn by the subject, but the embodiment is not limited thereto. For example, the blood pressure measurement device 10 may be an installation type device.

Further, in the embodiment, the case where the blood pressure value is dealt with as the biological information is described, but the embodiment is not limited thereto. For example, in addition to this, among pulses, body temperature, a blood glucose value, an electrocardiographic, and other biological information, the present invention may be applied to biological information that may be affected by a time (year, month, day).

Note that the present invention is not limited to the embodiment, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

A part or the entirety of the embodiment may be described as described in the following supplementary notes, but is not limited thereto.

### Supplementary Note 1

A biological information processing device, including:

a storage device configured to store biological information relating to a specified subject during a unit period including a plurality of times;

an estimation unit configured to estimate variable characteristics of the biological information based on the biological information relating to the specified subject that is stored;

an acquisition unit configured to acquire the biological information relating to the specified subject together with information indicating a measurement time of the biological information; and

a correction unit configured to correct the biological information relating to the specified subject that is acquired, based on the information indicating the measurement time that is acquired together with the biological information and the variable characteristics that are estimated.

### Supplementary Note 2

A biological information processing method, which is executed by a biological information processing device including a storage device, the method including:

storing biological information relating to a specified subject during a unit period including a plurality of times;

estimating variable characteristics of the biological information based on the biological information relating to the specified subject that is stored;

acquiring the biological information relating to the specified subject together with information indicating a measurement time of the biological information; and

correcting the biological information relating to the specified subject that is acquired, based on the information indicating the measurement time that is acquired together with the biological information and the variable characteristics that are estimated.

### REFERENCE SIGNS LIST

1 Storage unit
2 Acquisition unit
3 Estimation unit
4 Correction unit
5 Generation unit
10 Blood pressure measurement device
30 Portable terminal
50 Blood pressure processing device
70 Blood pressure evaluation device
11, 31, 51, 71 Control unit
12, 32, 52, 72 Storage unit
13, 33, 53, 73 Communication unit
14, 34 Operation unit
15, 35 Display unit
16 Blood pressure sensor
17 Acceleration sensor
18 Temperature/humidity sensor
36 GPS receiver
54, 74 Drive
55, 75 Storage medium
511 Acquisition unit
512 Variable characteristic estimation unit
513 Analysis unit
514 Output unit
515 Correction unit
516 Analysis information generation unit
521 Blood pressure processing program
721 Blood pressure evaluation program
522 Blood pressure change information
523, 722 Subject information
524 Variable characteristic estimation information
525 Variable characteristic statistic information
723 Evaluation reference information

1. A biological information processing device, comprising:
a processor; and
a memory, wherein
the memory is configured to store biological information relating to a specified subject during a unit period including a plurality of times,
the processor is configured to estimate variable characteristics of the biological information based on the biological information relating to the specified subject that is stored,
the processor is configured to acquire the biological information relating to the specified subject together with information indicating a measurement time of the biological information,
the processor is configured to correct the biological information relating to the specified subject that is acquired, based on the information indicating the measurement time that is acquired together with the biological information and the variable characteristics that are estimated,
the biological information includes first biological information associated with a first region and second biological information associated with a second region different from the first region,
the processor is configured to estimate first variable characteristics based on the first biological information, and estimate second variable characteristics based on the second biological information,
the processor is configured to further acquire position information indicating a measurement location of the biological information relating to the specified subject, and
the processor is configured to correct the biological information relating to the specified subject that is acquired, based on the first variable characteristics when the location information that is acquired is included in the first region or based on the second variable characteristics when the location information that is acquired is included in the second region.

2. The biological information processing device according to claim 1, wherein
the processor is configured to generate analysis information relating to the biological information relating to the specified subject, based on the biological information that is corrected by the processor and the variable characteristics that are estimated.

3. The biological information processing device according to claim 1, wherein
the processor is configured to update the variable characteristics that are estimated, based on the biological information relating to the specified subject that is acquired.

4. The biological information processing device according to claim 1, wherein
the processor is configured to estimate the variable characteristics of the biological information relating to the specified subject, further based on statistic information relating to variable characteristics of biological information relating to at least one unspecified subject during a unit period including a plurality of times.

5. The biological information processing device according to claim 1, wherein
the processor is configured to estimate a plurality of variable characteristics corresponding to different periods in the biological information, based on the biological information relating to the specified subject that is stored.

6. The biological information processing device according to claim 1, wherein
the unit period is set to any one of a year, a week, and a day.

7. The biological information processing device according to claim 1, wherein
the processor is configured to generate evaluation information corresponding to a health state of the specified subject, based on the biological information that is corrected by the processor.

8. The biological information processing device according to claim 7, wherein
the evaluation information includes at least one of information for determining acceptance of insurance purchase and information for calculating an insurance premium or reward points.

9. A biological information processing method, which is executed by a biological information processing device including a memory, the method comprising:
storing biological information relating to a specified subject during a unit period including a plurality of times;
estimating variable characteristics of the biological information based on the biological information relating to the specified subject that is stored;
acquiring the biological information relating to the specified subject together with information indicating a measurement time of the biological information; and
correcting the biological information relating to the specified subject that is acquired, based on the information indicating the measurement time that is acquired together with the biological information and the variable characteristics that are estimated, wherein
the biological information includes first biological information associated with a first region and second biological information associated with a second region different from the first region,
during the estimating, first variable characteristics are estimated based on the first biological information, and second variable characteristics are estimated based on the second biological information,
during the acquiring, position information indicating a measurement location of the biological information relating to the specified subject is further acquired, and
during the correcting, the biological information relating to the specified subject that is acquired is corrected based on the first variable characteristics when the location information that is acquired is included in the first region or based on the second variable characteristics when the location information that is acquired is included in the second region.

10. A non-transitory recording medium for recording a program, the program being configured to cause the processor included in the biological information processing device according to claim 1 to estimate, acquire and correct.

11. A non-transitory recording medium for recording a program, the program being configured to cause the processor included in the biological information processing device according to claim 2 to estimate, acquire, correct and generate.

12. A non-transitory recording medium for recording a program, the program being configured to cause the processor included in the biological information processing device according to claim 3 to estimate, acquire, correct and update.

13. A non-transitory recording medium for recording a program, the program being configured to cause the processor included in the biological information processing device according to claim 4 to estimate, acquire and correct.

14. A non-transitory recording medium for recording a program, the program being configured to cause the processor included in the biological information processing device according to claim 5 to estimate, acquire and correct.

15. A non-transitory recording medium for recording a program, the program being configured to cause the processor included in the biological information processing device according to claim 6 to estimate, acquire and correct.

16. A non-transitory recording medium for recording a program, the program being configured to cause the processor included in the biological information processing device according to claim 7 to estimate, acquire, correct and generate.

17. A non-transitory recording medium for recording a program, the program being configured to cause the processor included in the biological information processing device according to claim 8 to estimate, acquire, correct and generate.
